(19) United States
(12) Patent Application Publication
(10) Pub. No.: US 2022/0172841 A1
(43) Pub. Date: Jun. 2, 2022

(54) METHODS OF IDENTIFYING INDIVIDUALS AT RISK OF DEVELOPING A SPECIFIC CHRONIC DISEASE

(71) Applicant: Predicta Med Ltd, Herzliya (IL)

(72) Inventors: SHLOMIT STEINBERG-KOCH, Tel Aviv (IL); BENJAMIN GETZ, Ramat Hasharon (IL)

(73) Assignee: Predicta Med Ltd, Herzliya (IL)

(21) Appl. No.: 17/457,222

(22) Filed: Dec. 1, 2021

Related U.S. Application Data
(60) Provisional application No. 63/120,278, filed on Dec. 2, 2020.

(51) Int. Cl. G16H 50/20; G16H 10/60; G16H 20/10; G16H 20/30; G16H 20/60; G16H 20/70; A61B 5/00

(52) U.S. Cl. CPC G16H 50/20; G16H 10/60; G16H 20/10; G16H 20/30; A61B 5/7267; G16H 20/70; A61B 5/4088; A61B 5/7275; G16H 20/60

(57) ABSTRACT

Methods enabling prediction, screening, early diagnosis, and recommended intervention or treatment selection of chronic medical conditions using artificial intelligence operating in conjunction with large medical datasets. Logic is applied to historic population data to extract medical features and identify subjects with diagnosed chronic conditions, and the pre-diagnosis medical data is used to train a diagnosis classification algorithm. A self-supervised learning mechanism is separately used to generate a feature embedding transformation of the patient's medical history into representational feature vectors. These patient feature vectors together with their expected diagnoses are used to train a multi-label classifier model using supervised learning. The embedding transformation and the multi-label classifier are then applied to a current subject's data to generate a patient diagnosis probability vector, predicting the existence of chronic conditions. These methods are applied to diagnose progressive, chronic disorders in many different physiological systems.

### FIELD

The present invention relates to the field of predictive medical diagnosis, especially for use in screening, early detection, and treatment recommendation of chronic conditions.

### BACKGROUND

Chronic diseases affect millions of people worldwide. The prevalence is especially high in Western countries; nearly half of all Americans, 133 million, suffer from at least one chronic disease, and the number is growing. Chronic diseases such as heart disease, cancer, and diabetes are the leading causes of death and disability in the United States. Other chronic diseases may not be fatal, but may result in debilitating conditions. Chronic diseases have been reported to be on the rise around the world, making this category of diseases a public health crisis at unprecedented levels. Because of a lack of awareness of the underlying causes and difficulty in identifying multiple risk factors, these conditions are often undiagnosed until permanent physiological damage occurs. The multiple risk factors may comprise a wide variety of genetic susceptibilities, environmental conditions, behavioral habits, or as yet unidentified factors.

Providing the correct treatment for a chronic disease is also complex, and optimal treatment may heavily depend on genetic factors. To obtain proper treatment, subjects must visit a wide variety of specialties within medicine. Because chronic diseases affect multiple organs and systems in the body, teams of physicians in many specialties are often are needed to treat symptoms of an individual subject. This method of treatment is time consuming and often fiscally wasteful as there is typically no model for proper coordinated care amongst medical systems and physicians, needed to enable adequate monitoring, diagnostic testing and prescription drug treatments. Also, the addition of new cutting edge biologic treatments for patients requires an even higher level of coordination and expertise from physicians as these treatments, while revolutionary as lifesaving and quality of life-enhancing tools, must be heavily monitored for short-term and long-term adverse side effects and dosage issues.

Early signs and symptoms of chronic diseases are often vague and shared among a variety of conditions, such as gastrointestinal distress, elevated blood pressure or blood sugar levels, or coughing. Such non-specific symptoms have a wide range of differential diagnoses that often require a thorough clinical evaluation to diagnose. Prior means of predictive diagnosis have been attempted. However, these methods may be considered by some to be either limited in scope, or of limited effectiveness or convenience. There still exists a need for a more comprehensive solution for the screening and early diagnosis of chronic diseases which also provides a method to provide a treatment plan and thus overcomes at least some of the disadvantages of prior art systems and methods. There exists a need for a means of screening for chronic diseases that is rapid, specific, inexpensive, and provides the opportunity for preventive interventions to mitigate the long-term effects of these diseases.

Reference is made to the following patent-related documents:

U.S. Pat. No. 8,068,993 “Diagnosing inapparent diseases from common clinical tests using Bayesian analysis”, by V I Karlov, B Kasten, C E Padilla, E T Maggio, and F Billingsley, granted on 29 Nov. 2011, and assigned to Quest Diagnostics Investments LLC.

U.S. Pat. No. 7,877,343 “Open information extraction from the Web”, by M J Cafarella, M Banko, and O Etzioni, granted on 25 Jan. 2011, and assigned to the University of Washington.

US 2019/0087727 “Course of treatment recommendation system”, by J S Skellenger, published on 21 Mar. 2019, and assigned to Intermountain Intellectual Asset Management LLC.

CN 109920501 “Electronic health record classification method and system based on convolutional neural networks and active learning”, by A L Xiaotong, et al., and granted on 21 Jun. 2019.

These publications, and those mentioned other sections of the specification, are hereby incorporated by reference, each in its entirety.

### SUMMARY

The methods of the present disclosure are based on the ability to cluster individuals or groups of individuals based on defining characteristics, such as demographic, symptoms, lab test results, medications, procedures, biomarkers, or other measurable properties, while recognizing that individuals differ in an almost infinite number of characteristics representing their biologic individuality. The methods of the present disclosure collect, store, and analyze very large bodies of data to classify people according to their individual likelihood of acquiring symptoms of a specific chronic disease or having a specific chronic disease which is undiagnosed at the point of the data collection.

Because chronic diseases develop over time, during the first period of which affected individuals are clinically asymptomatic, and because genetic markers of heightened inherited susceptibility can be measured in genome-wide association studies often long before symptoms are noticed, identifying potential patients at an asymptomatic stage provides an opportunity to initiate preventive measures and minimize late-phase interventions which may be ineffective after irreversible tissue damage has occurred. Taken together with immunologic and biochemical markers, genomic markers can indicate that a potentially damaging process is in process long before symptoms occur, at a stage when intervention has a higher likelihood of preventing long-term damage.

The present disclosure describes new exemplary methods for predicting the risk in potential or latent patients, of the presence or the evolution of a chronic disease. Chronic disease in this disclosure refers to a disease that may be characterized by at least some of:

a) not congenital or apparently diagnosable at birth

b) typically has an adult onset

c) multifactorial or of unknown etiology

d) slowly progressive over at least one year

e) non-pathognomonic symptoms in the early phases of disease

f) requires ongoing medical attention, limits activities of daily living, or both.

Examples of such chronic diseases are Alzheimer disease and related types of dementia; arthritis; asthma; an autoimmune disorder; types of slowly progressive cancer such as ovarian cancer; chronic obstructive pulmonary disease (COPD) or other disease causing respiratory compromise; a circulatory disorder; Crohn disease; cystic fibrosis; diabetes; epilepsy; heart disease; HIV/AIDS; macular degeneration; mood disorders such as bipolar, cyclothymic, and depression; multiple sclerosis; pulmonary fibrosis; Parkinson disease, and numerous others.

The methods provide a screening recommendation for the general population according to relative risk, enable early diagnosis, and assist in formulating a treatment plan and disease management. The present disclosure describes a decision support platform, using artificial intelligence (AI) techniques such as machine learning, deep learning, and natural language processing (NLP) to enable early detection and personalized treatment selection.

Information for classifying individuals as being susceptible for, or having an early stage of, a specific disease may be collected from many different sources. Examples of such sources comprise the internet of things (IoT), a system of interrelated computing devices; mechanical and digital machines; objects, animals or people that are provided with unique identifiers and the ability to transfer data over a network without requiring human-to-human or human-to-computer interaction. Important patient-reported data related to non-specific early symptoms may be shared by subjects in informal, non-clinical settings. Such symptoms may be recalled in retrospect, and provide valuable sources of training data for machine learning. Thus, signs and symptoms predicting a given disease may be collected from social media such as disease-related support groups on Facebook; website forums for specific diseases; conference publications available online; and other published items of lay or scientific publications.

The novel algorithms of the present disclosure process a collection of subject data collected from sources comprising at least some of electronic medical records (EMR), electronic health records (EHR), and insurance claims data, suggest a subject's risk for having a common or uncommon chronic disease. Such clinical data may comprise height, weight, body mass index, blood pressure, oxygen saturation, and laboratory tests on biological fluids such as blood, urine, stool, or biopsy samples. Other sources of clinically relevant data may be gleaned from general sources of information that are widely used in a given population. Examples would be genetic polymorphisms in DNA that are linked with increased susceptibility to specific chronic diseases, or changes in RNA that may be linked to changes in gene expression and levels of proteins within a given tissue or cell type. Further examples comprise historical pharmacy records of prescriptions ordered and filled, information about the frequency of doctor or clinic visits, and specialist referrals. Frequency, duration and intensity of patient-reported symptoms may be gleaned from electronic records. Data may be gathered from widely used on-body sensors such as IoT sensors or monitors, and personal health application programs such as symptom trackers, which provide patient-specific physiological data or reported symptoms and habits. Memberships in gyms or health clubs may be used to identify mitigating or ameliorating factors in disease progression. Records of dietary supplements ordered, dietary interventions pursued, purchases at health food stores, restaurants may all be used to monitor the potential effect of diet on disease progression. Environmental influences from the ambient air or water may be collected from records of air travel and time spent in specific locales with higher or lower incidences of specific chronic diseases. The various sources of data comprise multiple parameters, which are compressed into a single feature or vector. This feature is representative of the tabular data used to generate the vector.

The method prioritizes subjects according to probability/risk and makes recommendations regarding the appropriate subsequent steps, such as related tests or prescription of a specific treatment. The disclosed process enables the system to recommend additional tests prior to establishing a firm diagnosis, so that the diagnosis and subsequent recommended treatment can be better assessed. The system provides explanatory output regarding relevant symptoms and signs, and analyzes trends, symptom recurrence, symptom distribution and all relevant patient history, to determine the risk of the particular subject having or developing the specific disease under consideration by the system. The service enables providers to seamlessly integrate this solution into their current workflow by either integrating the algorithms and software into the existing EMR system or by providing a separate software interface.

There are several advantages of the disclosed system and methods over previously disclosed methods. In contrast to U.S. Pat. No. 8,068,993 “Diagnosing inapparent diseases from common clinical tests using Bayesian analysis”, implementations of the present system are enabled to use the results of specific medical tests to trigger an early diagnosis, prior to clinical detection, rather than comparing posterior probabilities of the disease versus the non-disease conditions in Bayesian analysis to predict a current disease state. In further contradistinction to diagnosis using Bayesian analysis, implementations of the disclosed methods use machine learning algorithms with supervised learning, self-supervised representation learning, deep learning, and expert medical logic. Implementations of the machine learning and deep learning algorithms of the disclosed methods use context embedding of features and parameters, in contrast to U.S. Pat. No. 7,877,343 “Open information extraction from the Web”, and in contrast to the convolutional neural networks used by CN 109920501 “Electronic health record classification method and system based on convolutional neural networks and active learning”. In some implementations of methods of the present disclosure, the output enables not only diagnosis but treatment and follow up recommendations. The present system has an advantage over what is disclosed in US 2019/0087727 “Course of treatment recommendation system” in that the methods of the present disclosure are applicable to diagnosis, further testing, and follow-up planning, rather than only treatment recommendations. Apart from recommending treatments, some implementations of the disclosed methods may recommend genetic analysis of DNA or protein expression via RNA analysis to further personalize the subsequent treatment recommendations.

A summary of a further exemplary implementation of the disclosed methods to the diagnosis of a specific chronic disease comprises processing each individual's medical data set by the following steps:

(i) turning string-type data points into categorical data,

(ii) annotating every missing or censored data point,

(iii) allocating all missing or censored data points the median of non-missing data points for the relevant subpopulation,

(iv) creating a full data set for each individual,

(v) training said algorithm based on said full data sets,

(vi) providing a probability that a given individual will have a positive specific test,

(vii) validating said algorithm on a new data source,

(viii) choosing best hyper-parameters based on validated data sets, and

(ix) performing final evaluation on validated data sets.

In any such methods described in this disclosure, it is to be understood that the term predictive diagnosis is intended to cover also methods of screening for a chronic disease, or early detection of a chronic disease, or similar terms intended to relate to the determination of such a disease, whether present or whether expected to be present on the basis of the implementation of the methods.

### BRIEF DESCRIPTION OF THE DRAWINGS

The presently claimed invention will be understood and appreciated more fully from the following detailed description, taken in conjunction with the drawings in which:

FIG. 1 depicts the flow and processing of information for diagnostic, screening or decision support purposes in an exemplary implementation of the methods of the present disclosure;

FIG. 2 is a flow chart detailing a high level algorithm description for steps 101 to 104 in FIG. 1;

FIG. 3 is a flow chart detailing an exemplary high level algorithm description for celiac disease diagnosis, detailing a part of the flow chart of FIG. 1;

FIG. 4 depicts the flow and processing of information for intervention, treatment selection or therapeutic purposes in a representative implementation of the methods of the present disclosure;

FIG. 5 is a flow chart detailing a high level algorithm for the treatment model of FIG. 4;

FIG. 6 is a visualization of embedding space, illustrating the clustering together of subjects with similar historic medical records, as created by the self-supervised training process of the feature embedding model; and

FIG. 7 shows an exemplary implementation of a system structure used to carry out the methods described in FIGS. 1 to 5.

### DETAILED DESCRIPTION

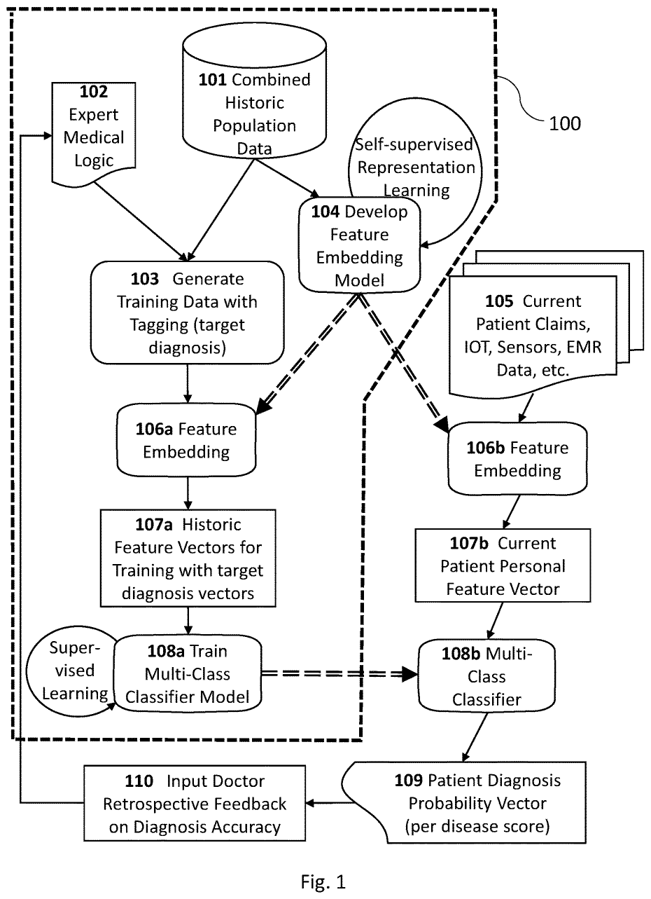

Reference is first made to FIG. 1, which illustrates schematically the overall structure of an exemplary implementation of the disclosed invention. A method detects individuals having characteristics that indicate a specific disease process. In a first phase of the method, historical patient data is collected from electronic medical records (EMR), electronic health records (EHR), insurance claims data, or data from other sources, such as symptoms relating to early disease manifestation from disease-related groups on social networks, or data from scientific literature. Data collection is followed by application of machine/deep learning, natural language processing (NLP), or other individual or combined machine learning techniques to train an algorithm of the method to identify subjects with the conditions which are to be diagnosed based on known cases of such disease in the historic population data. In a second phase, new patient data are input to the algorithm to enable determination of the probability and risk that a given individual in the new population has a chronic condition. Specifics of this process are delineated for an exemplary implementation of the method: in the example provided here, the method determines the probability of a given individual having a specific chronic disease with symptoms related to, for example abdominal perturbation, either currently or predicted to develop within a future time frame.

In block 101, a historic database of insurer medical claims and/or EMR data for a large population, representing the target population for this algorithm, is accessed to provide examples for training the models of the system. This data is augmented with additional sources, such as IoT sensor data, subject provided information, and aggregated statistics relevant to target subjects collected either from research datasets, or via use of the proposed system. This information is used in subsequent steps 103 and 106a to generate processed and filtered training information, ultimately for use in step 109.

In step 103, the large population data from block 101 is used in combination with rules derived from medical experts or known medical protocols, here referred to as “expert medical logic” 102, to generate tagged or labeled training data of subjects. Expert medical logic, entered into the system, is a database of rules providing specific logic how to classify subjects retrospectively, based on the data provided. This logic is based on interviews with medical doctors and information collected from research papers that enable the system to classify retrospectively who has been diagnosed with which diseases, so that this classification can be used to train the artificial intelligence classifier. Data tagging, in the context of this application, is the process of classifying and tagging data samples to label the historic population data with the target autoimmune diagnoses. The system uses the expert medical logic to retroactively identify and label each person's medical history with the autoimmune conditions that he has been later positively diagnosed with. The data is separated and a tag assigned to it prior to the diagnosis. The tagged training data will be used in subsequent steps to learn how to classify and predict the risk of having such conditions via analyzing patient data prior to the diagnosis.

In step 104, the large dataset of patient files is utilized to train a “feature embedding model”. The feature embedding model is a machine learning transformation that converts the patient data into a finite vector of real numbers. The vector space is of lower dimension than the entire patient data and therefore compresses the data keeping the important aspects and features that enable subject classification and diagnosis but also makes similar patients convert into vectors with a small distance between them. This transformation generates a representation of the data that is easier to classify and can better classify new subjects it has not seen before. This method is known as self-supervised representation learning and is used to generate an embedding model and optimize its parameters. Supervised learning and self-supervised representation learning are two different deep learning mechanisms. Supervised learning uses many classified examples to train the algorithm to correctly classify new samples based on multi-variate similarity to the training samples. Self-supervised learning is unsupervised learning in which the algorithm is trained to identify key differentiating features between classes of subjects, by going over many unclassified patient medical data files and studying the relationship between different segments or views of the medical files presented to it.

In this application, the embedding layer is a low-dimensional space for creating a dense encoding that represents the subject's medical history. This model is trained and generated using self-supervised learning and optimized over a large training set of historical medical data collected from a large population in step 101. The embedding for autoimmune disease diagnosis captures the semantics of the input from step 101, e.g., a variety of background data, comprising both medical data, environmental conditions, and patient risk factors, by placing semantically similar inputs close together in the embedding space. Although the embedding model itself may be reused among various populations, the subject population to which the method will be applied in steps 106b to 109 should be similar to, or derived from, the larger general population in block 101, such that the embedding parameters accurately distinguish among healthy individuals and those with a specific autoimmune diagnosis in that population. This is important because normal ranges of lab values and ways in which autoimmune conditions appear may differ among various populations. The embedding model parameters generated in step 104 are then input to step 106a to embed the tagged training data. The relevant patient data features selected for training are defined by current legacy methods, based on at least two of published medical literature, diseases registries, medical practice guidelines and the medical data.

In step 106a, the tagged history data of all of the recorded subjects, is passed through the feature embedding mechanism, loaded with the model derived in step 104, and is then converted into tagged feature vectors for training 107a.

In step 108a, a multi-output classifier model is trained using supervised learning of the tagged training data (107a). The steps 101 to 108a, shown in FIG. 1 within the dotted line 100, are steps used for the periodic training of the artificial intelligence models using the large historic population data. Steps 106b to 108b, on the other hand, are steps in which the feature embedding and classifying of the subject data are applied to the data of the currently analyzed patients, whose diagnoses are being resolved.

The output from step 108a comprises multi-label classifier model parameters, which are also used to classify current patient data vector 107b in step 108b. Multi-label classification is a classification mechanism that outputs multiple results associated with the likelihood of the inspected object being of a specified class. The classifier classifies object into multiple classes based on the input features of the object. In the context of this disclosure, the classifier provides probabilities of the analyzed person having: any autoimmune disease, any gastrointestinal autoimmune disease, or a specific autoimmune disorder, based on features found in his collection of medical records and data.

The embedding model parameters output from the self-supervised learning in step 104 are also used as input model for step 106b. Additional input for step 106b comprises raw data on a current subject's present situation and recent history from a variety of sources. The raw data may comprise at least some of patient insurance claims, electronic medical record data, and information gleaned or acquired from IoT, sensors, and health app data 105. In this step, the system applies the embedding parameters developed in step 104 to the raw data from block 105 and the output is a personal feature vector 107b representing the data of the current subject. This output is then used as the input for the multi-label classifier model of step 108b.

In step 108b, the model parameters developed in 108a are used to classify the personal feature vector (107b).

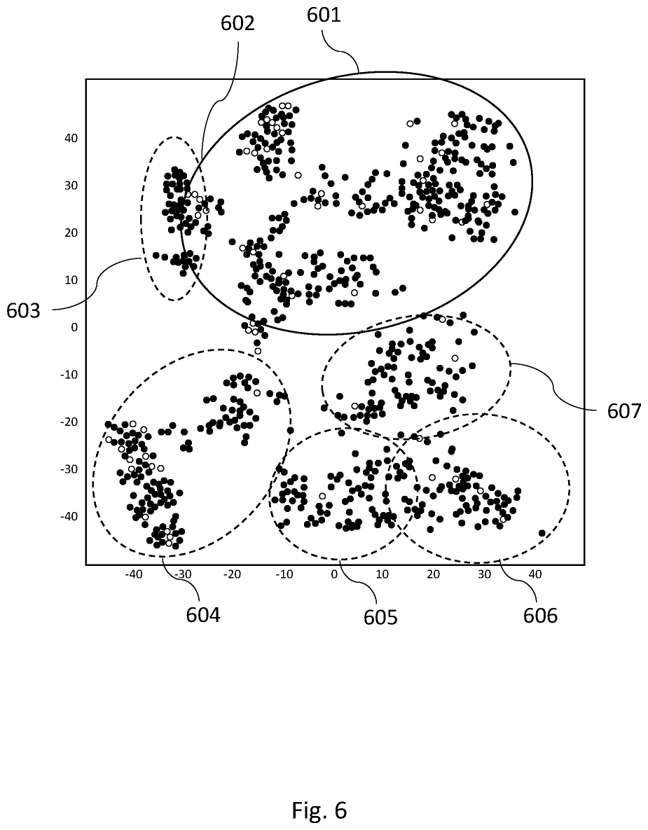

Step 109 uses the output from step 108b to generate a corresponding diagnosis probability vector with multiple values associated with a patient's file, that provides a probability that the current subject has each condition analyzed, such that further diagnosis recommendations and treatment recommendations can be derived. Each value in the vector corresponds to one of the autoimmune conditions that the system is programmed to seek, with individual values representing the likelihood of the person having the associated autoimmune disease or condition. Usually, the system will compare these values to a threshold for exceeding or going below the pre-defined normal range, and when the threshold has been crossed, suggesting the possibility of a disease state, the system will generate an indication or alert. This process is explained in more detail in FIG. 6.

In the event that no diagnosis is made, step 109 may also provide output indicating the likelihood that the given individual may develop a chronic disease in the future.

Finally, in step 110, the doctor or other health care provider, generates retrospective feedback on the diagnostic accuracy of the output generated by the system. The physician's analysis of the system's performance is input to the expert medical logic database of step 102, to update and improve that data.

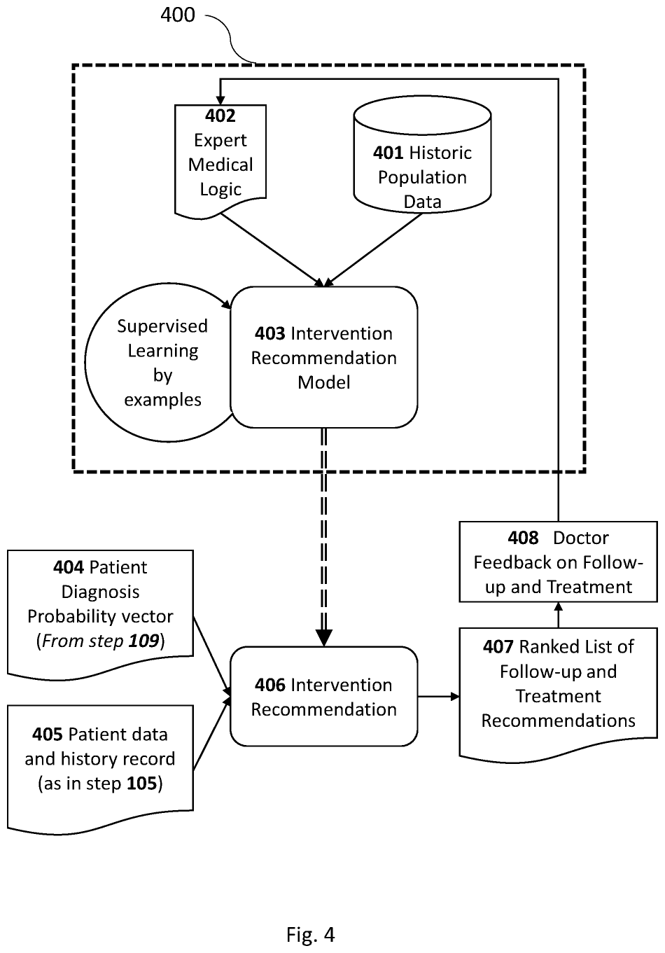

In other implementations of the disclosed methods, the algorithm is able to provide from steps 109 and 110, treatment recommendations, referral suggestions, or follow-up advice, as will be further delineated in FIG. 4.

The following general parameters used for diagnosis of at least one chronic disease refer to the process described in FIG. 1. Examples of parameters or features from the patient's data file, used in the machine learning algorithm, may fall into the following categories: demographics including family history of the given chronic disease, symptoms, concurrent diagnoses, lab tests, medications, procedures, and current and past measurements such as BP, height, weight, and BMI. A large number of parameters may be used in training the algorithm; over time, additional, different, or fewer parameters may be incorporated to improve the diagnostic accuracy of the method. Each of these categories are further defined and detailed below. Additional categories and additional parameters within each category may be included over time as the machine learning algorithm identifies and correlates other factors as having relevancy to the diagnosis of a specific chronic disease. Demographics includes gender, birth season, and age at the time of the test and, if known, age at the time of the disease diagnosis.

Symptoms included are collected from the patient's historical data up to a predefined time window, before medical diagnosis of this condition actually took place for that patient. Specific relevant symptoms comprise those relating to the system primarily affected by the subject's symptoms. Further symptoms may be included over time as the algorithm improves its specificity and accuracy, and is able to incorporate additional symptom patterns and correlate them with the diagnosis of the specific chronic disease.

The algorithm uses a cluster of diseases that have overlapping clinical presentations to define a differential diagnosis, or list of possible disease diagnoses based on the clinical presentation. Risk factors for a given disease in the differential diagnosis are determined for each patient based on at least some of the patient's gender, age, genetic background, history of medications, family history, and for women, gynecological history.

Laboratory tests such as CA-125 have limited value in the diagnosis of, for example, ovarian cancer, are thus used in combination with other parameters. The sensitivity of CA-125 in distinguishing between benign and malignant masses ranges between 61% and 90%, while specificity ranges between 35% and 91%. As a screening modality, such a test has more value if measured sequentially over time and in older women. Measurements such as height, weight, and BMI include the minimum values, maximum values, and the first and last in the predefined time, e.g., 5 years, preceding the examination. For children, growth measurements over time are an important input to the system. The selected laboratory blood tests have relevance for diagnosis of diseases in the differential diagnosis. Further laboratory tests of the blood or other body fluids may be included over time as the algorithm improves its specificity and accuracy, and is able to incorporate additional lab values and correlate them with the diagnosis of a specific chronic disease.

Clinical manifestations and other parameters that may be included in the algorithm comprise age of symptom onset, symptoms such as pain of a specific quality (burning, sharp, dull, prickly, searing) and intensity (on a scale of 1-10), and in a discrete distribution (along a nerve root, in a pattern typical for referred pain, localized or diffuse). Other symptoms depend on the physiological system affected, and may comprise pressure, bleeding or discharge, gastrointestinal disturbance; cough; tremor; weakness; and many others. Para-clinical findings may comprise results of laboratory tests; imaging studies such as X-ray, CT, ultrasound, and MRI; and immuno-histopathology on a biopsy sample. History of medications prescribed and other drugs taken are also included. Further medications and other routes of administration may be included over time as the algorithm improves its specificity and accuracy, and is able to incorporate additional findings and correlate them with the specific diagnosis.

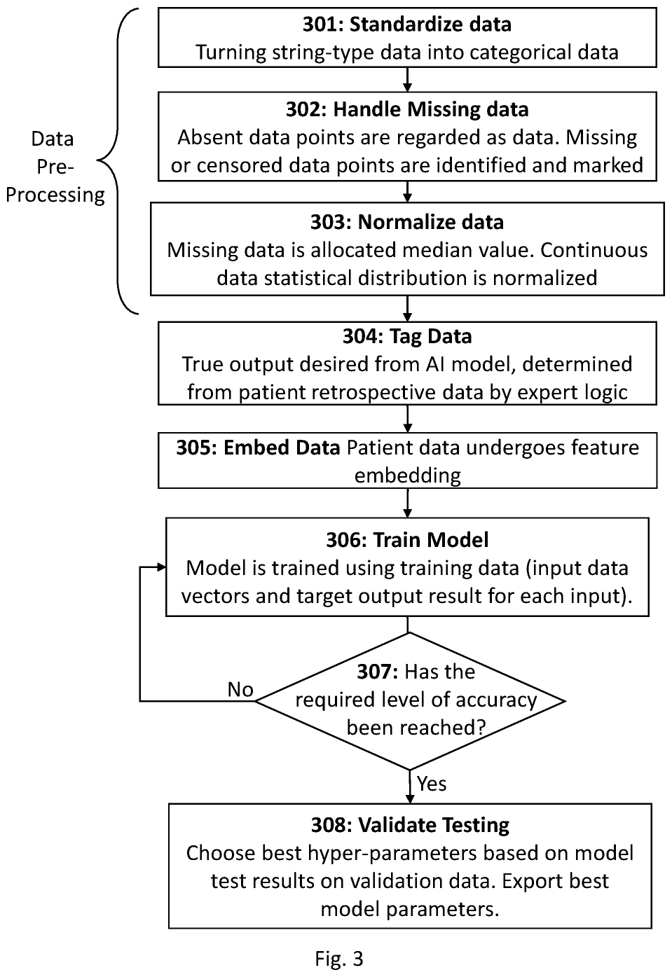

Objective measurements or values derived from measurements included in the algorithm comprise height (decrease in percentile, based on the z-score); weight; weight loss; and BMI. As with other parameter categories described above, further measurements may be included over time as the algorithm improves its specificity and accuracy, and is able to incorporate additional findings and correlate them with the specific diagnosis. In the initial iterations of the algorithm as it is being trained, inclusion criterion for subjects as having a specific diagnosis can be based on the current standard of care for that diagnosis. The following section explains the procedures used in the use of expert medical logic and the tagging of patients based on the historic data, as will be shown implemented in step 304 of FIG. 3 hereinbelow.

Based on these initial results, subjects are divided into two groups. The treatment group is comprised of those individuals having one or a set of positive test results, or, in the event that there are no positive test results, similar indications mentioned above for insurance claims data; the control group is comprised of those having normal values for the test results. Subjects who reach the diagnostic criteria for having the specific disease are used to establish the ‘ground truth’, i.e., results of patients who have been historically diagnosed with that disorder. Ground truth refers to a dataset with accurate tagging that is used to train the model and test it, as the expected result is known to be accurate. In implementations of the present disclosure, ground truth is generated from the historic patient data files by identifying those files that have clear indication of positive diagnosis of specific diseases or clear indication of no disease. The system separates those files into data collected at a predefined time prior to the time of diagnosis and into target diagnosis tagging that embodies the correct diagnosis as later found for that subject.

In cases where specific diagnostic test results are not available, e.g. insurance claims without lab results, the ‘ground truth’ can be defined by identifying cases where a specific diagnosis of, for instance, suspected celiac disease, appears in the claims data at a later time after procedures or tests related to such a diagnosis have been performed.

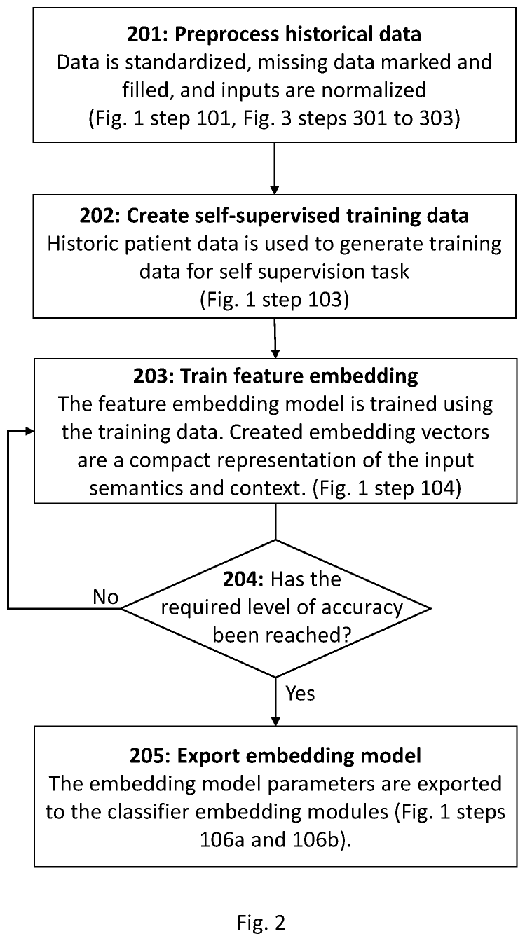

FIG. 2 provides further details of the machine learning and other artificial intelligence procedures incorporated in the feature embedding model developed in steps 101 to 104 of FIG. 1.

In step 201, data are input from a large historic database of different medical, health and claims data collected per subject of a large population. These data are pre-processed to standardize, normalize and remove/fill missing values, a process that enhances the quality and quantity of information available to use for training, and upon which to base subsequent decisions.

In step 202, the input data is processed to generate training data for a self-supervised task. These tasks may include prediction of parts of the patient record based on another known part of that record, identifying randomly added, changed or removed data points in the medical record, or similar tasks that enable the model to learn a compact representation of the input data file via a smaller vector of real numbers. These patient data vectors are optimized in such a way that information located in proximity in the embedding space represents a similar level of risk with respect to the diagnostic probability of a given subject for developing the autoimmune disease under consideration.

In step 203, this embedding model is trained on a very large data set with self-supervised target outputs, its output providing the parameters for the embedding model. In other words, the embedding model transformation parameters are optimized so that the embedding vectors created will represent in a compact way the data features needed for diagnosis.

In step 204, the method determines if the required level of accuracy has been reached by measuring the accuracy achieved in the self-supervised training tasks. If not, the method returns to step 203 and refines the parameters with additional optimization cycles. If the required level of accuracy has been reached, the method proceeds to step 205, wherein the system exports the embedding model parameters to the classifier embedding modules in FIG. 1, steps 106a and 106b.

Reference is now made to FIG. 3, which explains the data handling procedures shown in the previous drawings in further detail, using an exemplary implementation of the method for predicting and diagnosing a given disease. The algorithm details sub-steps specifically for determining the probability of a given individual to have a positive result indicating, for example, reduced pulmonary function or an autoimmune disorder. It is to be understood that the same process may be applied to other medical data with predictive value for a given autoimmune disease, such as lab values, genetic biomarkers, or imaging studies.

Steps 301 to 303 delineate individual steps used in treatment of historical data from FIG. 1 step 101 and FIG. 2 step 201. Step 305 relates to the periodic training of the artificial intelligence model 100 in FIG. 1; similarly, the output of step 307 corresponds to the application of the multi-label classifier parameters derived in FIG. 1 step 108a to the individual subject data in step 108b.

In step 301, string-type data is standardized to categorical data. A string is a data type used in programming that is used to represent text rather than numbers, comprised of a set of characters. In this application, the word “autoimmune” and the phrase “gastrointestinal autoimmune disorder” are both strings. By contrast, categorical data have a limited, and usually fixed, number of possible values, e.g., assigning each individual to a particular group, such as “normal”, “celiac disease”, or “at risk for celiac disease”, on the basis of the diagnosis probability vector. Data is collected from a source such as EMR, or from other sources such as a survey that is completed by the individuals or by an application such as the Apple Health App, which electronically collects health-related data from other applications and sources.

In step 302, each data point is annotated as present, missing or censored. Missing information is then used as data during the model learning by noting its absence in a separate feature and taking a median value for that data point from among all data sets in the relevant population, which comprises the data source. Features which comprise the algorithm inputs are determined, and cutoff values are selected for being outside the normal range and indicating a possible diagnosis of a given chronic disease.

In step 303, all missing or censored data points are allocated the median of non-missing data points to complete the data set without changing its distribution. Features with continuous values (e.g. lab test numeric values) are normalized based on their common distribution in the population.

In step 304, which corresponds to step 103 of FIG. 1, the system uses expert medical logic to retroactively tag the historic data of each subject according to all autoimmune diseases that have been later diagnosed for this patient (based on more recent data collected). Using the diagnosis tagging, the system creates training vectors based on the historic data (prior to diagnosis), which, when added together with the correct diagnosis tagging, represents the desired classifier output.

In step 305, new subject data is entered and undergoes feature embedding. The embedding transformation converts the long vector of input features into a smaller embedding vector using the embedding model parameters from step 205. The results are training vectors, in which patients with similar conditions related to autoimmune diseases have similar vectors, making the training phase more efficient. An exemplary graph illustrating training vectors and new patient vectors is further delineated in FIG. 6.

In step 306, which corresponds to the periodic training steps, 100, of the artificial intelligence model in FIG. 1, the algorithm is trained and tested iteratively using supervised learning of the tagged training vectors and testing on the control group or validation set, as described in the periodic training steps of the artificial intelligence model 100 of FIG. 1, until the algorithm performs satisfactorily; the results should match the ground truth results according to the sensitivity and specificity pre-defined for the diagnosis classifier.

In step 307, the method determines if the required level of diagnostic accuracy has been reached; if not the method continues the supervised learning process of 306. If the required level of diagnostic accuracy has been reached, the method proceeds to step 308.

In step 308, the model is tested and validated using validation training samples set aside for the validation phase. The best model hyper-parameters, chosen to optimize the system performance using designated training vectors, are selected based on the validation set results, and the final performance evaluation is performed on a preselected test set. Hyperparameters, in machine learning, are structural parameters of the algorithm whose values are set before the learning process begins. By contrast, the values of other AI model parameters, sometimes called weights or factors in neural network architectures, are derived via training. Both of these types of ‘parameters’ are in distinction to the medical parameters or clinical features, referred to elsewhere in the present disclosure, that are used to define a subject's susceptibility or probability of developing a specific autoimmune disease.

Reference is now made to FIG. 4, a schematic representation of an implementation of the method for interventional recommendations. The steps within the dotted line 400 represent periodic training of artificial intelligence models. In block 403, an intervention recommendation model is developed, using supervised learning by examples. The training inputs for this model are examples generated from the population medical record database 401 using medical guidelines 402, and by collecting patients' response to specific treatments and scoring them accordingly. The information in steps 401 and 402 may be the same or different as that in FIG. 1 steps 101 and 102. These scores are used as target results to train the algorithm. After the model 400 is developed through machine learning or other form of artificial intelligence, the recommendation model parameters are input into the intervention recommendation model 406. Other inputs to the model 406 are the patient diagnosis probability vector from step 110 in FIG. 1, and patient historical data 405, comprised of previous tests and procedures, which may be the same data as provided in FIG. 1, step 105. The output of the intervention recommendation model is a ranked list of follow-up and/or treatment recommendations in step 407. Additionally, to the routine output in step 407, in step 408, the doctor or other health care provider can input retrospective feedback on the diagnostic accuracy of the output generated by the system. This information is used to improve the expert medical logic in step 402.

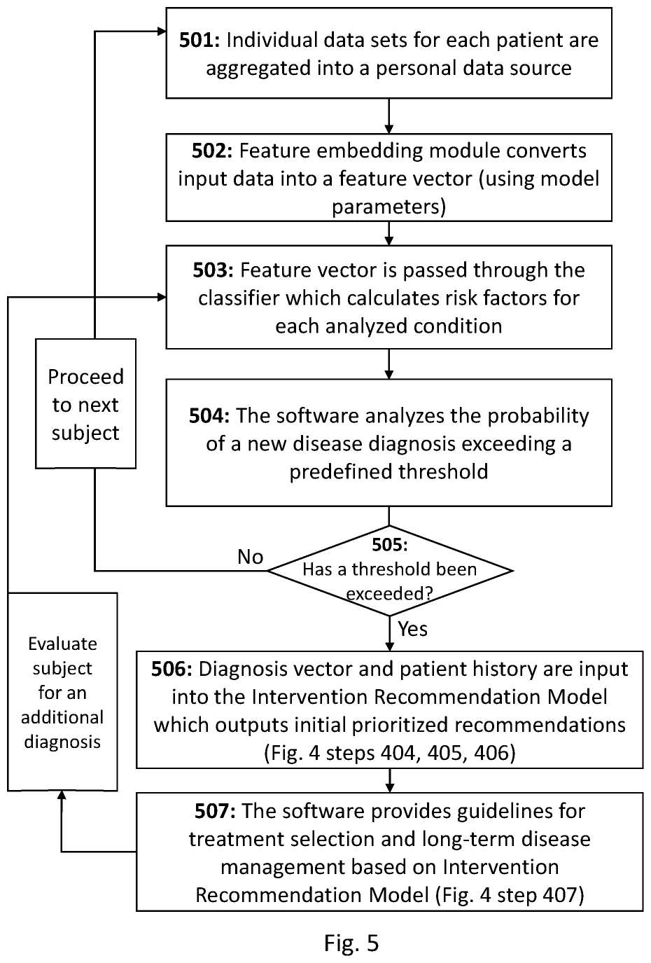

Reference is now made to FIG. 5, showing a description of how the algorithm operates within the full diagnostic system. Once the algorithm is fully trained and validated as described in FIG. 3, it may be used on other populations of undiagnosed individuals for screening and detection purposes. In representative implementations, the algorithm calculates the probability of each given individual to have a positive result of a given test suggestive or pathognomonic for a specific chronic disease, and notifies the software operator of cases reaching a specific threshold of probability, as described below. Image processing of, for example, small intestinal biopsy tissue slides from individuals with a high predictive probability of having or developing celiac disease may be used to compare with images from individuals having previously been given this diagnosis using small intestinal biopsy.

In step 501, individual data are aggregated into a personal patient data source. In step 502, the algorithm analyzes or processes each patient data set. In step 503, the algorithm calculates the probability of each subject having a disease in the category of, for example, respiratory insufficiency, autoimmune, or mental illness, by integrating the vectors for beyond-threshold values of any number of tests that fall outside the normal range. At this step, if active learning is used, the system may indicate need for additional medical information or request additional data from the subject. Active learning is a machine learning training method where the algorithm provides questions or suggests collection of additional data in order to improve its ability to provide specific and accurate diagnosis. The method analyzes the input patient vector to be classified, and if the vector falls in a “gray area” where the diagnosis is not clear, it will request additional information or data, such as for instance, a lab test result or a question to the subject about missing data. Following input of answers to these requests, the algorithm will be in a better position to provide a clear and more probable diagnosis.

In step 504, the system provides an alert when the probability of a given subject having one of the defined gastrointestinal autoimmune diseases, exceeds a predefined threshold. If the user requests more details, the system can provide explainability analysis of its decision, by means of identifying important parameters leading to its diagnosis decision. Explainability refers to mechanisms of analyzing the operation of machine learning, or other types of AI-based decision support algorithms, and presenting to the user how the recommendation has been reached and what parameters have most influenced this decision. The goal of these mechanisms is to build trust in the system's correctness by enabling an expert user to trace the decision factors and logic of the results and also enables effective human oversight of the process.

In step 505, the method determines whether a new diagnosis has been made. If not, the method proceeds to analyze the data of the next subject by returning to step 501. If a new diagnosis has been made, the method proceeds to step 506, in which the system provides initial guidelines for intervention selection among a group of available treatment options, and based on prior training of the algorithm for optimal outcomes. Such intervention may be based on novel therapies developed by third parties, which are expected to be developed over time. Thus, the system may be updated on a regular basis to incorporate the current standard of treatment for a specific chronic disease. Thus, the outcomes should continually improve over time. In step 507, the system provides guidelines for chronic disease supervision based on algorithm training. Such guidelines may provide short- or long-term follow-up recommendations, goals for exercise, diet, medical treatment, and other advice for successful long-term management of the condition and minimization of secondary complications.

In the case of evaluating the likelihood that a subject has any one of a number of chronic diseases with a conglomeration of non-specific symptoms comprising the differential diagnosis, the method takes into account the risk factors present in the subject, age, gender, family history, symptom duration, and other factors (. . . ), and uses these factors to sift and rank the most likely diagnoses. The basis of personalizing the treatment selection is based on results of different patient subpopulations and groups. For example, lab results, concurrent diagnoses, and symptom clusters of specific diseases tend to differ between adult and pediatric populations.

The disclosed algorithm and system are able, via iterative processing and machine learning, to identify and distinguish between classical and non-classical presentations of a specific disease. A further ability of the algorithm and system is to identify silent or asymptomatic disease, that may only manifest physiological damage at a later time point. Such patients are unaware of compromised capacity of the affected physiological system(s) and do not complain of symptoms, which may be mild, but nevertheless experience damage to their small intestine resulting in villous atrophy. Studies show that despite reporting no symptoms, after going on a strict gluten-free diet these individuals report better health and a reduction in acid reflux, abdominal bloating and distention and flatulence.

Reference is now made to FIG. 6, showing a visualization of the embedding space, to illustrate the clustering of subjects with respect to lab values or other exemplary indicators of autoimmune disease. The data illustrate implementation of feature embedding, a machine learning method in which a large multi-dimensional set of features is converted into a smaller dimensional space containing the relevant information of the original data. In this example, feature embedding allows construction of a more efficient and accurate classifier for autoimmune diagnosis that generalizes from the reference population in which diagnoses of autoimmune diseases have been made, to as yet unseen new patient populations. The embedding vector captures semantics of the input by placing semantically similar inputs closer together in the embedding space, as illustrated and described below.

The graph is an output of the T-SNE (t-distributed stochastic neighbor embedding) algorithm, which is a dimensional reduction method that may be used to visualize data set clustering. Specifically, the algorithm takes high-dimensional data and visualizes them in a low-dimensional space of two or three dimensions. In this two-dimensional graph, the x- and y-axes represent transformed parameters that visually represent the similarities and dissimilarities between different inputs or points, each having a mean positioned at zero and deviations extending in both positive and negative directions from the mean. From these representations, it is possible to differentiate the clusters/groups and therefore predict, based on an individual subject's embedded feature vector, if he/she has or is likely to develop a condition under consideration. The distribution in two dimensions of training data points for the transformed parameters in a given population is represented by black dots, whereas new patient data points are shown in empty dots, as explained further below. The larger, general population with normal values for the measured parameters are shown in the central-lower region of the graph, illustrating a range of normal values for the given parameters. By contrast, individuals diagnosed with a specific disease have values that differ significantly from normal and are part of the distinct clusters outlined by dotted ovals. These smaller disease clusters represent individuals having values that fall far from the mean average of normal individuals in the general population for the measured parameter on the y-axis, i.e., above the normal threshold. In terms of diseases affecting the respiratory system, each small cluster 603 to 607 may represent, for example, individuals identified as having or being predisposed to develop, one of asthma, chronic obstructive pulmonary disease (COPD), lung cancer, cystic fibrosis, pulmonary fibrosis, sleep apnea or an occupational lung disease such as asbestosis. Thus, even though all of the individuals in these disease clusters have values outside the normal threshold for the parameter measured on they-axis, such as lung capacity, they vary among each other in terms of the second parameter represented on the x-axis, such a peak flow on expiration as measured in pulmonary function tests, and each cluster or diagnosis can thus be distinguished from the others. Because in mild cases values of measured parameters for an individual may fall at the edge of the normal range 601, in a region of overlap 602 between normal and pathologic, such individuals would be flagged and their record identified for further follow up at specific intervals of time.

The new subjects' data points, shown as empty dots, appear throughout the parameter range and cluster together with similar subjects from the training set, so the classifier algorithm is able to use the clustering to suggest the correct diagnosis for such patients. The transformation of the feature vectors into the embedding space allows the system to predict or diagnose an individual at risk of a given autoimmune disease by placing this subject close to others with similar parameter values, i.e., sharing the same signs, symptoms, and other diagnostic criteria.

Individuals who have been screened and have a probability of a specific diagnosis that is above normal but fails to reach threshold can be monitored with additional visits to follow the course of signs and symptoms over time, to determine whether the threshold is reached that would transfer the individual from the normal group to the treatment group.

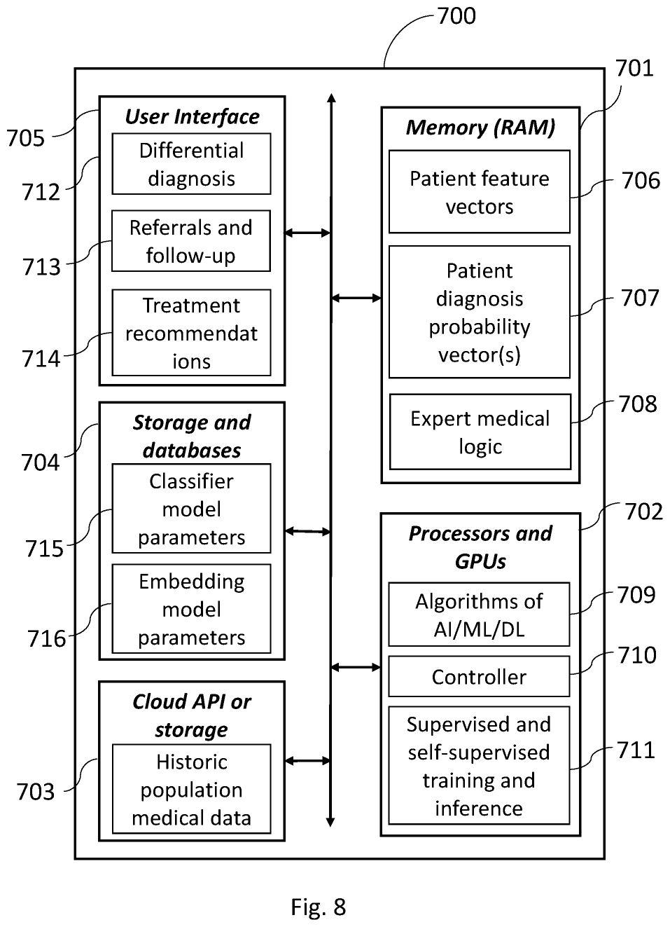

Reference is now made to FIG. 7, showing a schematic representation of the system structure 700 used to perform the methods described herewithin above. In this disclosure, the term system may refer to, be part of, or comprise a computing system, storage, connectivity to patient health data via local databases or remote application programming interfaces; include an application specific integrated circuit (ASIC); a digital, analog, or mixed analog/digital discrete circuit; a digital, analog, or mixed analog/digital integrated circuit; a combinational logic circuit; a field programmable gate array; at least one processor 702 (shared, dedicated, or group) that executes code; memory 701 (shared, dedicated, or group) that stores code executed by a processor 702; other suitable hardware components, such as optical, magnetic, or solid state drives, that provide the described functionality; or a combination of some or all of the above, such as in a system-on-chip.

The term code, as used above, may include software, firmware, and/or microcode, and may refer to programs, routines, functions, classes, and/or objects. The term shared processor encompasses a single processor that executes some or all code from multiple modules. The term group processor encompasses a processor that, in combination with additional processors, executes some or all code from one or more modules. The term shared memory encompasses a single memory that stores some or all code from multiple modules. The term group memory encompasses a memory that, in combination with additional memories, stores some or all code from one or more modules. The term memory may be a subset of the term computer-readable medium. The term computer-readable medium does not encompass transitory electrical and electromagnetic signals propagating through a medium, and may therefore be considered tangible and non-transitory. Non-limiting examples of a non-transitory tangible computer readable medium include nonvolatile memory, volatile memory, magnetic storage, and optical storage.

The apparatuses and methods described in this disclosure may be partially or fully implemented by one or more computer programs executed by one or more processors 702. The computer programs include processor-executable instructions that are stored on at least one non-transitory tangible computer readable medium, i.e., memory 701. The computer programs may also include and/or rely on stored data 703, 704.

In some implementations, the system comprises a memory 701, processors and graphic processing units 702, cloud application program interface or storage 703, other storage and databases 704, and a user interface 705. The components of the system 700 are further delineated below, with reference to the steps of the exemplary method in FIG. 1 to which they correspond. The memory 701 may comprise data relating to patient feature vectors 706 (FIG. 1, steps 106a, 106b), patient diagnosis probability vectors 707 (FIG. 1, step 109), and expert medical logic 708 (FIG. 1, step 102). The processing unit 702 may comprise algorithms of artificial intelligence, machine learning, and deep learning 709, a controller 710, and supervised and self-supervised training and inference 711 (FIG. 1, steps 103, 104, 106a, 106b, 108a, 108b). The cloud storage 703 may comprise historic population medical data (FIG. 1, step 101, 105). The at least one database 704 may comprise the data incorporating classifier model parameters 715 and embedding model parameters 716. The user interface 705 communicates with the medical staff or other professionals using the system, and provides the output of the system, such as a diagnosis or list of possible diagnoses, ranked in order of likelihood 712, referrals to specialists and follow-up guidelines 713, and in some implementations, treatment recommendations or guidelines 714.

In some implementations, the user interface is configured to communicate with other systems and share information via the IoT and other tools. The system may be configured to provide alerts to doctor or to insurer system or even to the subject via health app or other patient interface. Furthermore, the system may be configured to receive feedback from the user or a doctor regarding the accuracy of the classifier model results. Such human feedback regarding diagnosis or treatment/follow-up recommendations may be incorporated in order to influence future training cycles of its models, such as is shown in step 110 of FIGS. 1 and 408 of FIG. 4.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure.

It is appreciated by persons skilled in the art that the present invention is not limited by what has been particularly shown and described hereinabove. Rather the scope of the present invention includes both combinations and sub-combinations of various features described hereinabove as well as variations and modifications thereto which would occur to a person of skill in the art upon reading the above description and which are not in the prior art.

We claim:

1. A method for predictive diagnosis of at least one chronic disease in a subject, comprising:
(i) applying to health related data of the subject, a machine learning method adapted to convert parameters of the health related data, some of which may be indicative of a diagnosis of at least one chronic disease, into a vector that provides a compact representation of the health related data that reflects a medical condition of the subject; and
(ii) applying a classifier model to the vector generated in step (i) to identify whether the medical condition of the subject indicates a likelihood of the subject having or developing one or more chronic diseases,
wherein the classifier model incorporates supervised learning and at least one of expert medical logic or self-supervised representation learning, and is generated by:
(a) accessing a database comprising records of health related data of a large population;
(b) tagging at least most of the records with information indicating if a member of the large population with whom a record is associated, has been diagnosed with the at least one chronic disease;
(c) performing the machine learning method on at least some of the tagged health related records, to convert tagged records into target diagnosis vectors indicating that the member associated with the tagged record has been diagnosed with the at least one chronic disease;
(d) training the classifier model iteratively to relate features of each target diagnosis vector with a previous diagnosis of the at least one chronic disease by correlating parameters of the tagged records representing features of a chronic disease for the member associated with that record; and
(e) repeating the training until the correlation of parameters with the diagnosis of a chronic disease shows a desired level of accuracy, such that application of the classifier model to the vector generated in step (i) predicts with the desired level of accuracy, the likelihood that the subject has the at least one chronic disease.

2. A method according to claim 1, wherein a chronic disease may be at least one of a slowly progressive chronic disease, such as Alzheimer disease and related types of dementia; arthritis; asthma; an autoimmune disorder; types of slowly progressive cancer such as ovarian cancer; chronic obstructive pulmonary disease (COPD) or other disease causing respiratory compromise; a circulatory disorder; Crohn disease; cystic fibrosis; diabetes; epilepsy; heart disease; HIV/AIDS; macular degeneration; mood disorders such as bipolar, cyclothymic, and depression; multiple sclerosis; pulmonary fibrosis; Parkinson disease, and numerous others.

3. A method according to claim 1, wherein the classifier model is trained to predict a diagnosis of either a chronic disease affecting a specific physiological system, or a specific chronic disease.

4. A method according to claim 1, wherein the machine learning method is developed using self-supervised representation learning.

5. A method according to claim 1, wherein the vector providing a compact representation of the health related data of the subject is generated using context embedding.

6. A method according to claim 5, wherein a database comprising records of health related data of a large population is used to generate the context embedding.

7. A method according to claim 1, wherein the database comprises historical data on a subpopulation of subjects, some of whom have a diagnosis of the at least one chronic disease.

8. A method according to claim 1, wherein tagging the records is performed using expert medical logic.

9. A method according to claim 1, wherein the multi-class classifier model is trained using supervised learning.

10. A method according to claim 1, wherein the same database is used for generating both the machine learning method and the classifier model.

11. A method according to claim 1 wherein the predicted diagnosis of a chronic disease in the subject is validated by a health practitioner.

12. A method according to claim 1 wherein the health related data of the subject is tagged and added to the database comprising records of health related data of the large population.

13. A method according to claim 12 wherein feedback from the health practitioner is appended into the expert medical logic to improve accuracy of the predictive diagnostic method.

14. A method according to claim 1, wherein the parameters are defined by current legacy methods based on a least one of published medical literature, diseases registries, medical practice guidelines and said medical data.

15. A method according to claim 1, wherein the health-related data of the large population is derived from at least some of electronic medical or health records, the internet of things or other sensor data, health application feeds, social media, expert medical logic, and medical claims.

16. A method according to claim 1, wherein training the classifier model is performed using at least one of artificial intelligence, machine learning, deep learning, natural language processing, reinforcement learning, and big data analytics techniques.

17. A method according to claim 1, wherein the classifier model is a multi-label classifier model that outputs multiple results associated with the likelihood of the subject having more than one specific type of chronic disease.

18. A method according to claim 1, further comprising: using supervised learning, training an intervention recommendation model to provide at least one of recommended intervention, treatment selection, disease management recommendations, and decision support guidelines.

19. A method according to claim 19, wherein the intervention recommendation model is trained by supervised learning from at least one of either the success or the effectiveness of interventions and treatments in the database comprising records of health related data of a large population.

20. A method according to claim 1, wherein the subject belongs to a subpopulation of the large population whose records of health related data comprise the database.

21. A method according to claim 1, wherein the health related data of the large population database are pre-processed by standardizing, marking and filling missing data points, and normalizing inputs.

22. A method according to claim 22, wherein the health related data of the large population database are used to create self-supervised training data.

23. A method according to claim 23, wherein the training data are used to train the machine learning method used to create embedding vectors that are a compact representation of the input semantics and context.

24. A method according to claim 1, wherein the health related data of the large population database are standardized by turning string-type data into categorical data.

25. A method according to claim 1, wherein missing data are handled by identification, marking, and filling in absent data points as actual data.

26. A method according to claim 26, wherein absent data points are allocated a median value, and the statistical distribution of continuous data is normalized.

27. A method according to claim 1, wherein optimal hyper-parameters are chosen and exported based on model test results on validation data.

28. A method according to claim 1, wherein the machine learning method is a feature embedding transformation.

29. A method according to claim 1, wherein the tagging of the records is also performed with information indicating with which chronic disease the subject has been diagnosed.

30. A method according to claim 1, wherein application of the classifier model to the vector generated in step (i) predicts with the desired level of accuracy, the likelihood that the subject has the at least one specific chronic disease.

31. A method according to claim 1, wherein the health related data of the subject comprises at least some of: genetic sequence data, sensor health data; frequency of health care visits; subject-reported symptoms; pharmacy prescriptions; vital signs and measurements such as height, weight, body mass index, blood pressure, oxygen saturation, heart rate, and temperature; laboratory test results; histopathology results; imaging study results; and on-body or remote sensor feeds.

32. A method according to claim 1, further comprising:
applying an intervention recommendation model to the patient diagnosis probability vector, if the subject is identified as having greater than a pre-defined likelihood of having or developing a chronic disease,
wherein the intervention recommendation model is generated by:
a) accessing a database comprising records of health related data of members of a large population;
b) using expert medical logic to determine most effective treatment and follow up parameters of members of the large population who have been previously diagnosed with and treated for a chronic disease; and
c) training the intervention recommendation model iteratively to provide model parameters that meet accuracy requirements on test inputs, the model parameters provided by the intervention recommendation model being applied to the health related data of the subject and the patient diagnosis probability vector, to generate recommended interventions.

33-49 (canceled)

50. A system for predictive diagnosis of at least one chronic disease in a subject, comprising:
i) at least one processor comprising a controller adapted to run at least one of artificial intelligence algorithms, and training and inference logic;
ii) a memory adapted to enable the processor to access expert medical logic and at least one of patient feature vectors and patient diagnosis probability vectors stored on the memory; and
iii) at least one type of data storage adapted to contain records of health related data of a large population, classifier model parameters, and embedding model parameters derived from the training of the artificial intelligence algorithm by the processor,
wherein the at least one processor is configured to:
a) apply the expert medical logic to the health related data to produce updated patient feature vectors and patient diagnosis vectors;
b) generate classifier model parameters based on algorithm training to process the feature vectors;
c) input the classifier model parameters into an embedding model to classify the patient diagnosis vectors; and
d) output the likelihood of a predictive diagnosis of at least one chronic disease in the subject.

51-54. (canceled)
